(19) United States
(12) Patent Application Publication
(10) Pub. No.: US 2018/0079670 A1
(43) Pub. Date: Mar. 22, 2018

(54) LIQUID PROCESSING METHOD AND LIQUID PROCESSING DEVICE

(71) Applicant: SHIBATA CO., LTD., Shinjuku-ku (JP)

(72) Inventors: Ryo TANAKA, Hachioji-shi (JP); Noriko MARUYAMA, Machida-shi (JP)

(73) Assignee: SHIBATA CO., LTD., Shinjuku-ku (JP)

(21) Appl. No.: 15/565,750

(22) PCT Filed: Apr. 12, 2016
(86) PCT No.: PCT/JP2016/061841

§ 371 Date: Oct. 11, 2017

(30) Foreign Application Priority Data
Apr. 13, 2015 (JP) 2015-081794

(51) Int. Cl. C02F 3/34; C02F 3/12; C12Q 1/04

(52) U.S. Cl. CPC C02F 3/34; C02F 3/12; C02F 2101/32; C02F 2303/04; C12Q 1/04

(57) ABSTRACT

A liquid processing method and a liquid processing device capable of processing a liquid with optimal microorganisms, and responding swiftly even when changes occur in the liquid. The method includes processing in a biological treatment tank that houses microorganisms, and a microorganism optimization step of optimizing the microorganisms used in the biological treatment tank. The microorganism optimization includes a liquid extraction step of extracting a liquid that includes a stock solution discharged from a liquid handling system; a screening step of screening optimal microorganisms suited for processing performed in the biological treatment tank using the liquid that includes the stock solution as a culture substrate; a culturing step of culturing the screened optimal microorganisms; and a supply step of supplying the cultured optimal microorganisms into the biological treatment tank.

### FIELD OF THE INVENTION

The present invention relates to a liquid processing method and a liquid processing device, and more particularly to a liquid processing method and a liquid processing device that use optimal microorganisms for a liquid to be processed.

### BACKGROUND ART

Techniques for treating wastewater and the like using microorganisms included in activated sludge or the like have been widely used to date. As a processing method that uses microorganisms, a method in which processing is performed using microorganisms that have been locally grown and naturally produced since the formation of the earth, that is, indigenous microorganisms, has been generally used. In the processing method that uses indigenous microorganisms, organic and inorganic matter included in a liquid to be processed serves as a source of nutrients for the microorganisms. The microorganisms included in activated sludge gradually decrease the organic and inorganic matter, ultimately removing the organic and inorganic matter.

The activated sludge process is a technique that makes it possible to decompose and remove various substances included in water in a short period of time. The activated sludge process is, a method that has the erect of reducing the biochemical oxygen demand (BOD) and chemical oxygen demand (COD) defined in the Water Pollution Control Law in Japan.

However, the activated sludge process has several problems.

The first problem is that the microorganisms used in the activated sludge process are not always the optimal microorganisms for the liquid to be processed. That is, the microorganisms consume the organic and inorganic matter included in the liquid to be processed as a nutrient source. Thus, when microorganisms adapted to the organic and inorganic matter included in the liquid are not included in the activated sludge, the liquid cannot be efficiently processed using the microorganisms.

The second problem is that the activated sludge process is extremely vulnerable to factors that cause fluctuation in the external environment. In general, when a group of microorganisms that concentrate at high density is formed, such as with activated sludge, the speed of transition to microorganisms adapted to the environment is extremely slow, resulting in low responsiveness to sudden changes in the external environment. Thus, once the state of the activated sludge collapses, a significant amount of time (several weeks to one month or longer) is required until the group of microorganisms once again becomes a stable state. As a result, when some kind of fluctuation occurs in a stock solution subject to liquid processing, the microorganisms used in the processing until then are not suited for the processing after the fluctuation.

Attempts at quick recovery when the state of the activated sludge collapses have been proposed.

For example, in the technique proposed in Patent Document 1, in addition to operating conditions, such as aeration intensity, an artificially cultured microbial preparation is secondarily added.

The third problem is that certain substances included in the processing target cannot be processed. That is, when chemically stabilized substances, oils and fats, biological growth inhibitors (antibiotics), and the like are included in the wastewater to be processed, there has been a limit to processing by the activated sludge process. In the activated sludge, there exist microorganisms that have the capability to decompose various substances and utilize the decomposed substances as a nutrient source. However, these microorganisms do not always preferentially exhibit such a function. The types of microorganisms included in the group of microorganisms that constitute the activated sludge are not constant. The types of microorganisms that preferentially function under each condition change according to various factors, such as drainage load, water quality, temperature, amount of dissolved oxygen, and pH.

General wastewater is substantially made up of compounds that are readily decomposed by biological activity. Thus, only the microorganisms that decompose these compounds preferentially function and become stabilized. As a result, even if microorganisms capable of processing low-concentration substances and difficult-to-decompose substances included in the wastewater exist, these microorganisms are buried without ever exhibiting processing capacity.

In response to these problems, the technique of Patent Document 2 temporarily separates the microorganisms capable of decomposing organophosphorus compounds, which are difficult-to-decompose substances, from the activated sludge. In this technique, after the separated microorganisms are cultured, the wastewater that includes the organophosphorus compounds is processed by adding the microorganisms to the activated sludge once again.

### PATENT DOCUMENTS

Patent Document 1: Japanese Laid-Open Patent Application No. 2011-200765

Patent Document 2: Japanese Laid-Open Patent Application No. 2002-301494

### SUMMARY OF THE INVENTION

### Problems to be Solved by the Invention

Nevertheless, in the technique proposed in Patent Document 1, the effect of the microbial preparation is significantly impacted by the quality and the environment of the injected wastewater, and thus it is not always possible to sufficiently exhibit the expected effect.

On the other hand, in the technique proposed in Patent Document 2, when the wastewater to be processed changes in quality or the like due to external factors, the changed wastewater cannot be processed.

The present invention was made to resolve the above-described problems, and it is therefore an object of the present invention to provide a liquid processing method and a liquid processing device capable of firstly processing a liquid using optimal microorganisms for the liquid, and secondly responding swiftly even when some kind of change occurs in the liquid.

### Means for Solving the Problems

(1) The liquid processing method according to the present invention for solving the above-described problems is a method that comprises a step of processing a liquid in a biological treatment tank that houses microorganism and processes a liquid discharged from a liquid handling system that handles the liquid. The liquid processing method according to the present invention further comprises a microorganism optimization step of optimizing the microorganisms used in the biological treatment tank to obtain optimal microorganisms, the microorganism optimization step comprising a liquid extraction step of extracting a liquid that includes a stock solution discharged from the liquid handling system, a screening step of screening the optimal microorganisms suited for processing performed in the biological treatment tank using a liquid that includes the stock solution as a culture substrate, and a culturing step of culturing the screened optimal microorganisms.

According to this invention, the liquid is processed using a liquid processing method comprising the steps described above, making it possible to perform processing using microorganisms adapted to each liquid handling system that handles the liquid. In particular, this invention comprises the screening step of screening the optimal microorganisms suited for processing performed in the biological treatment tank using a liquid that includes the stock solution discharged from the liquid handling system as the culture substrate. Thus, according to this invention, it is possible to culture microorganisms adapted, to the liquid handled by the liquid handling system and process the liquid using the microorganisms.

In the liquid processing method according to the present invention, the liquid processing method further comprises a supply step of supplying the cultured optimal microorganisms into the biological treatment tank.

According to this invention, the supply step of supplying the cultured optimal microorganisms into the biological treatment tank is included, making it possible to house the optimal microorganisms applicable to the liquid to be processed in the interior of the biological treatment tank.

In the liquid processing method according to the present invention, the liquid processing method further comprises a primary processing step of incorporating the stock solution discharged from the liquid handing system and performing predetermined processing, and a secondary processing step of processing the liquid after primary processing that was subjected to the predetermined processing in the biological treatment tank. In the liquid extraction step, the stock solution is directly extracted from the liquid handling system or the liquid is extracted at any position between the liquid handling system and an inflow port of the biological treatment tank.

According to this invention, the primary processing step described above is further included, making it possible to make the stock solution discharged from the liquid handling system into a liquid suited for processing in the secondary processing step performed using the biological treatment tank. Further, in the liquid extraction step, the stock solution is directly extracted from the liquid handling system or the liquid is extracted at any position between the liquid handling system and the inflow port of the biological treatment tank, making it possible to culture microorganisms suited for the secondary processing step performed using the biological treatment tank.

In the liquid processing method according to the present invention, the microorganism optimization step further comprises a microorganism extraction step of extracting microorganisms from, among the biological treatment tank and an external environment other than the biological treatment tank, at least the biological treatment tank. Then, in the screening step, the microorganisms extracted in the microorganism extraction step are added to the culture substrate.

According to this invention, the microorganisms used in the secondary processing step are extracted from, among the biological treatment tank and an external environment other than the biological treatment tank, at least the biological treatment tank. With a high probability that the microorganisms suited for the liquid processed in this biological treatment tank will survive up to this point in the biological treatment tank, it is possible to swiftly screen the microorganisms suited for the liquid to be processed.

In the liquid processing method according to the present invention, the screening step adds the microorganisms extracted in the microorganism extraction step to the culture substrate, cultures the microorganisms, and then selects the fast-growing microorganisms from among the added microorganisms.

According to this invention, fast-growing microorganisms are selected. Inorganic and organic matter contained in the culture substrate is considered a nutrient source most suited for the fast-growing microorganisms. Thus, by selecting the fast-growing microorganisms, it is possible to screen the optimal microorganisms for the liquid to be processed.

In the liquid processing method according to the present invention, the microorganism optimization step further comprises a timing determination step of determining the timing at which the screening step is executed.

According to this invention, the timing at which the screening step is executed is determined in the timing determination step, making it possible to screen the microorganisms in an appropriate time period.

In the liquid processing method according to the present invention, the timing determination step promotes execution of the screening step periodically or when a given physical property determined in advance in the stock solution or the liquid extracted in the liquid extraction step reaches a control level set in advance.

According to this invention, the timing determination step promotes execution of the screening step as described above, making it possible to swiftly screen the microorganisms adapted to the liquid to be processed even after changes occurred in the water quality of the liquid, and perform appropriate processing.

In the liquid processing method according to the present invention, the microorganism optimization step can be executed a plurality of times according to the timing determined in the timing determination step. In the screening step, microorganisms screened in a most recent screening step executed at a most recent timing determined in the timing determination step, and microorganisms cultured in the microorganism optimization step executed earlier than the most recent timing are compared. Then, the liquid processing method further comprises a microorganism selecting step of selecting microorganisms suited for processing in the secondary processing step at the most recent timing.

According to this invention, the microorganism selecting step described above is included, making it possible to select whether the microorganisms screened in the screening step executed at the most recent timing or the microorganisms screened at an earlier screening step are appropriate microorganisms. Thus, the present invention makes it possible to process the liquid using the optimal microorganisms.

In the liquid processing method according to the present invention, the screening step includes a plurality of culturing steps of repeatedly housing the culture substrate and the microorganisms in a screening container and culturing the microorganisms.

According to this invention, with the screening step including the plurality of culturing steps as described above, it is, possible to reliably screen the optimal microorganisms while the culturing step is repeated.

In the liquid processing method according to the present invention, the microorganism optimization step further comprises a disinfection processing step or a sterilization processing step of disinfecting or sterilizing the stock solution or the liquid extracted in the liquid extraction step.

According to this invention, the disinfected or sterilized stock solution or liquid can be used as the culture substrate, making it possible to prevent obstruction of microorganism growth in the microorganism optimization step.

(2) The liquid processing device according to the present invention for solving the above-described problems comprises a biological treatment tank that houses microorganisms and is for processing a liquid. This liquid processing device is a device that processes a liquid discharged from a liquid handling system that handles the liquid. The liquid processing device further comprises a microorganism optimization processing part for optimizing microorganisms used in the biological treatment tank to obtain optimal microorganisms. The microorganism optimization processing part comprises a liquid extracting part that extracts a liquid that includes a stock solution discharged from the liquid handling system, a screening processing part that screens the optimal microorganisms suited for processing performed in the biological treatment tank using the liquid that includes the stock solution as a culture substrate, and a culture processing part that cultures the screened optimal microorganisms.

According to this invention, it is possible to perform processing using microorganisms adapted to each liquid handling system that handles liquid. In particular, this invention comprises the screening processing part that screens the optimal microorganisms suited for processing performed in the biological treatment tank using the liquid that includes the stock solution discharged from the liquid handling system as the culture substrate, making it possible to culture microorganisms adapted to the liquid handled by the liquid handling system and process the liquid using the microorganisms.

In the liquid processing device according to the present invention, the liquid processing device further comprises a supply pan that supplies the cultured optimal microorganisms into the biological treatment tank.

According to this invention, the supply part that supplies the cultured optimal microorganisms into the biological treatment tank is included, making it possible to house the optimal microorganisms adapted to the liquid to be processed in the interior of the biological treatment tank.

The liquid processing device further comprises a primary processing part that incorporates the stock solution discharged from the liquid handling system, performs predetermined processing, and supplies the liquid after the predetermined processing to the microorganism treatment tank, and the liquid extracting part directly extracts the stock solution from the liquid handling system or extracts the liquid at any position between the liquid handling system and an inflow port of the biological treatment tank.

According to this invention, the primary processing part described above is further included, making it possible to make the stock solution discharged from the liquid handling system into a liquid suited for processing in the biological treatment tank. Further, in the liquid extracting part, the stock solution is directly extracted from the liquid handling system or the liquid is extracted at any position between the liquid handling system and the inflow port of the biological treatment tank, making it possible to culture microorganisms suited for processing in the biological treatment tank that is performed using the biological treatment tank.

The screening processing part comprises a sterilization processing part for sterilizing the stock solution or the liquid.

According to this invention, it is possible to use the disinfected or sterilized stock solution or liquid as a culture substrate, making it possible to prevent obstruction of microorganism growth in the microorganism optimization processing part step.

### Effect of the Invention

According to the present invention, it is possible to firstly process a liquid using optimal microorganisms for the liquid, and secondly respond swiftly even when some kind of change occurs in the liquid.

### BRIEF DESCRIPTION OF THE DRAWINGS

FIG. 1 is a summary view illustrating a summary of steps of a liquid processing method according to the present invention.

FIG. 2 is a configuration diagram illustrating an outline of a liquid processing device according to the present invention.

FIG. 3 is a summary view illustrating a summary of a step of optimizing microorganisms that is included in the liquid processing method according to the present invention.

FIG. 4 is a configuration diagram illustrating an outline of a microorganism optimization processing part of the liquid processing device according to the present invention.

### EMBODIMENTS OF THE INVENTION

The following describes an embodiment of the present invention with reference to the drawings. Note that the technical scope of the present invention is not limited to only the descriptions and drawings below.

### [Basic Step of Liquid Processing Method]

A liquid processing method according to the present invention is a method that comprises a step of processing a liquid in a biological treatment tank that houses microorganisms, and processes a liquid discharged from a liquid handling system that handles the liquid. The most basic step of this liquid processing method comprises a microorganism optimization step of optimizing, microorganisms used in the biological treatment tank. This microorganism optimization step comprises a liquid extraction step of extracting a liquid that includes a stock solution discharged from the liquid handling system that handles the liquid to be processed, a screening step of screening optimal microorganisms suited for processing performed in the biological treatment tank using the liquid that includes the stock solution as a culture substrate, and a culturing step, of culturing the screened optimal microorganisms. In this specification, the microorganisms optimized in the microorganism optimization step are referred to as “optimized microorganisms” and, conversely, “optimized microorganisms” refers to microorganisms optimized in the microorganism optimization step.

### [Basic Configuration of Liquid Processing Device]

A liquid processing device according to the present invention is a device that comprises a biological treatment tank that houses microorganisms and is for processing a liquid, and processes a liquid discharged from a liquid handling system that handles the liquid. The most basic configuration of this liquid processing device comprises a microorganism optimization processing part for optimizing microorganisms used in the biological treatment tank. The microorganism optimization processing part comprises a liquid extracting part that extracts a liquid that includes a stock solution discharged from the liquid handling system, a screening processing part that screens optimal microorganisms suited for processing performed in the biological treatment tank using the liquid that includes the stock solution as a culture substrate, and a culture processing part that cultures the screened optimal microorganisms. In this specification, the microorganisms optimized in the microorganism optimization processing part are referred to as “optimized microorganisms” and, conversely, “optimized microorganisms” refers to microorganisms optimized by the microorganism optimization processing part.

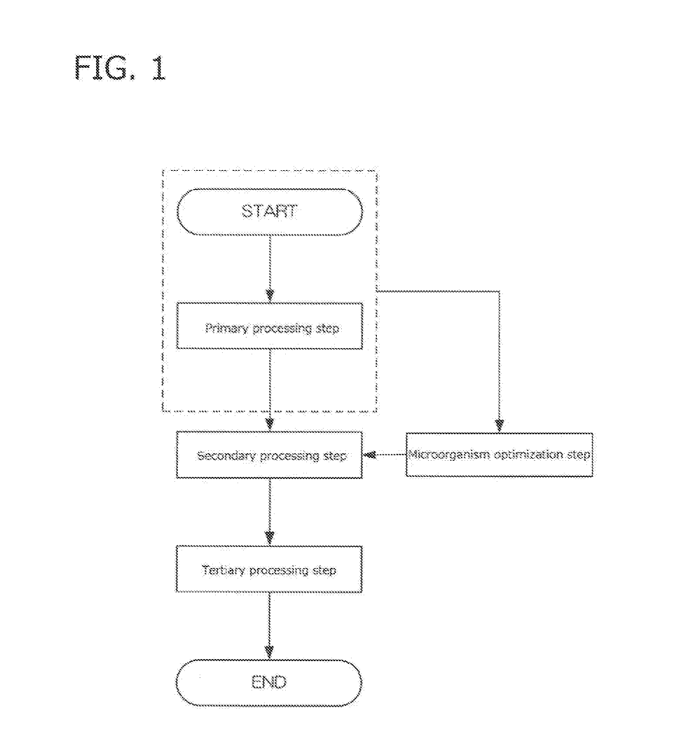

The overall flow of the liquid processing method according to the present invention will now be described with reference to FIG. 1. The liquid processing method, as illustrated in FIG. 1, comprises a primary processing step of incorporating a stock solution discharged from a liquid handling system (not illustrated) and performing predetermined processing, a secondary processing step of processing the liquid after primary processing that was subjected to the predetermined processing in a biological treatment tank that houses the microorganisms, and a microorganism optimization step of optimizing the microorganisms used in the processing of the liquid after primary processing.

Note that, in FIG. 1, the starting point of the arrow that extends toward the microorganism optimization step is the section of the dashed line that surrounds an upstream side of the secondary processing step and includes “START.” The meaning of this is to clearly indicate that this process is on the upstream side of the secondary processing step and includes the timing at which the stock solution is produced in the liquid handling system.

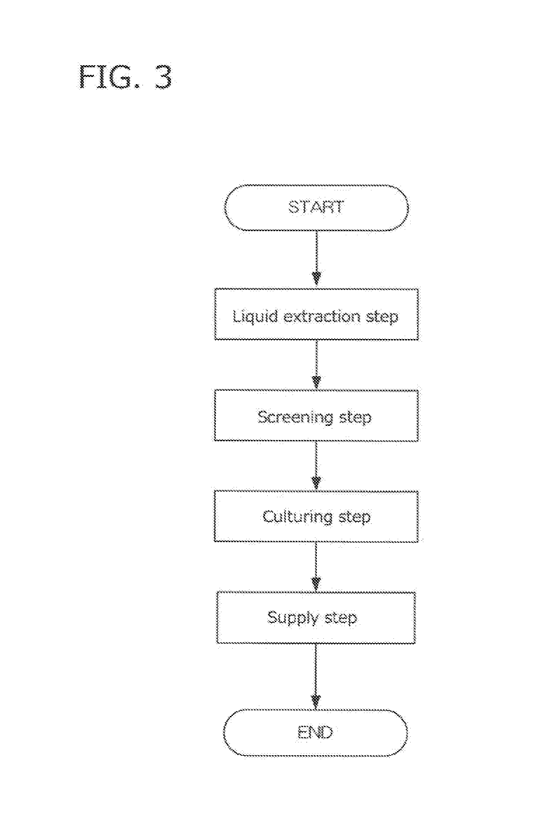

The microorganism optimization step comprises a liquid extraction step, a screening step, a culturing step, and a supply step (refer to FIG. 3).

The liquid extraction step is a step of directly extracting the stock solution from the liquid handling system or extracting the liquid at any position between the liquid handling system and an inflow port of the biological treatment tank. The screening step extracts the stock solution extracted in the liquid handling system or the liquid at any position between the liquid handling system and the inflow port of the biological treatment tank. The screening step screens the optimal microorganisms suited for processing the liquid after primary processing using the extracted liquid as a culture substrate. The culturing step is a step of culturing the screened optimal microorganisms. The supply step is a step of supplying the cultured optimal microorganisms into the biological treatment tank.

Further, the microorganism optimization step according to the present invention is a step of optimizing the microorganisms used when biological processing is performed using biological treatment tanks 21, 22, 23 on the liquid after the stock solution discharged from the liquid handing system that handles the liquid was subjected to the primary process, as illustrated in FIG. 3. The microorganism optimization step extracts the stock solution, the liquid undergoing primary processing, or the liquid after primary processing, and establishes the solution or liquid as a culture substrate. The microorganism optimization step comprises the screening step. The screening step adds microorganisms to the culture substrate, and screens upper-level fast-growing microorganisms from among the added microorganisms.

Note that the content not processed in the primary processing step and the secondary processing step described above is processed by a tertiary processing step (refer to FIG. 1) described later.

According to the liquid processing method and liquid processing device of the present invention, it is possible to firstly process a liquid using the optimal microorganisms for the liquid, and secondly respond swiftly even when some kind of change occurs in the

Note that, in this specification, the fluid that flows through the liquid handling system and the fluid discharged from the liquid handling system are referred to as “stock solution.” The liquid incorporated into a primary processing part 10 and undergoing processing by the primary processing part 10 is referred to as the “liquid undergoing primary processing.” The liquid after being subjected to predetermined processing by the primary processing part 10 is referred to as the “liquid after primary processing,”

### [Liquid Processing Method and Liquid Processing Device]

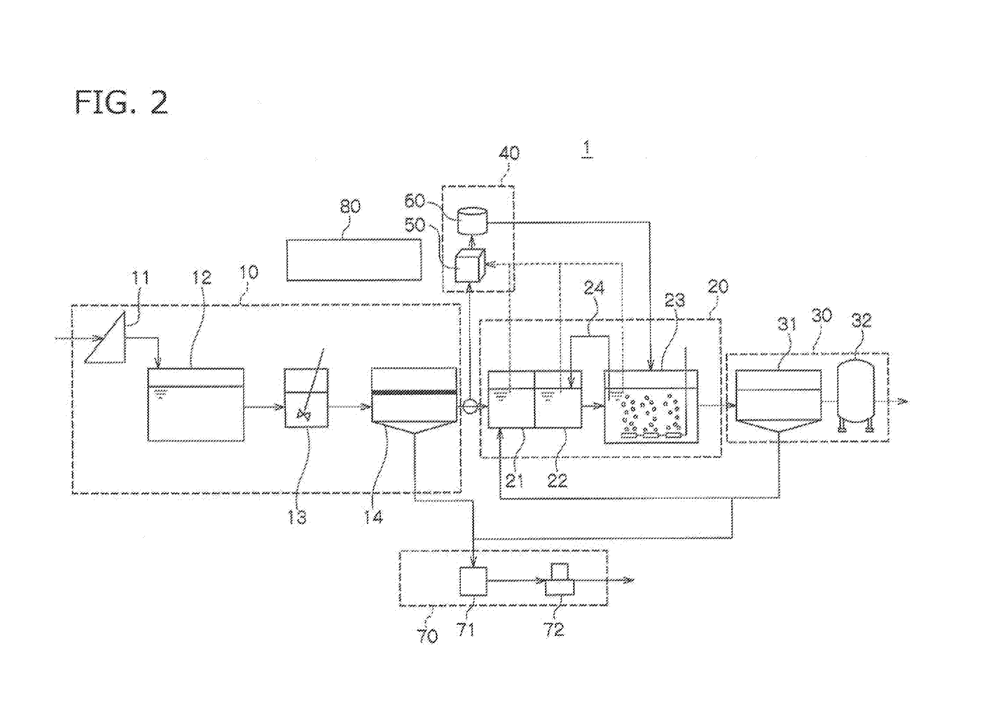

The liquid processing method is performed using, for example, a liquid processing, device 1 illustrated in FIG. 2. This liquid processing device 1, as illustrated in FIG. 2, comprises the primary processing part 10, a secondary processing part 20, and a microorganism optimization processing part 40. The microorganism optimization processing part 40 comprises a screening processing part 50 and a culture processing part 60. Further, the liquid processing device 1 comprises a tertiary processing part 30 on a downstream side of the secondary processing part 20. The liquid processing device 1 comprising such processing parts executes the primary processing step, the secondary processing step, and the tertiary processing step in each processing part, as illustrated in FIG. 1. Further, the liquid processing method comprises the microorganism optimization step executed by the microorganism optimization processing part 40 provided to the liquid processing device. This microorganism optimization step is a step of screening and culturing the optimal microorganisms for processing the liquid to be processed. Moreover, the liquid processing device 1 comprises a sludge discharging part 70 for discharging sludge produced from the primary processing part 10, the secondary processing part 20, and the tertiary processing part 30. Each component illustrated in this FIG. 2 will now be specifically described with reference to the drawings as appropriate.

### [Primary Processing Part]

The primary processing part 10 executes the primary processing step described above. This primary processing part 10 is a processing part that is connected to the liquid handling system (not illustrated) that discharges the stock solution processed by this liquid processing device 1, and first incorporates and processes the stock solution. The processing step executed by the primary processing part 10 is the primary processing step. This primary processing part 10 is configured by arranging a screen part 11, an adjustment tank 12, a reaction tank 13, and an aggregate removal tank 14, in that order from upstream. In this primary processing part 10, predetermined processing is performed on the liquid. Specifically, the primary processing part 10 performs processing that removes relatively large contaminants included in the liquid to be processed, processing that maintains a constant flow rate and adjusts the pH value of the liquid to be processed by the liquid processing device 1, processing that injects a liquid chemical to floccculate the liquid and settles the flocculated floc or causes the flocculated floc to float, and the like.

### (Screen Part)

The screen part 11 performs, among predetermined processing performed in the primary processing step, processing that filters relatively large contaminants included in the stock solution. This screen part 11 is disposed just behind the inlet of the stock solution, and is positioned furthest upstream from the liquid processing device 1. The screen part 11 selected is a screen having a structure corresponding to the type of liquid to be processed and contaminant to be filtered from among a bar screen, a scraping screen, an inclination-type wire screen, and the like.

Note that the bar screen is a screen that comprises a filtering part in which a plurality of bars are arranged in parallel. The scraping screen is a screen configured to immerse the filtering part in a flow path of the liquid and scrape the liquid using a rake. The inclination-type wire screen is a screen in which a filtering plate formed by wire is disposed on an incline with respect to the flow path, causing the liquid to flow down from the filtering plate.

### (Adjustment Tank)

The adjustment tank 12 performs, among the predetermined processing performed in the primary processing step, processing that feeds the liquid to processing performed in downstream steps at a constant flow rate. This adjustment tank 12 maintains a constant flow rate of the liquid incorporated into the liquid processing device 1. This adjustment tank 12 comprises a tank having a relatively large capacity. The adjustment tank 12 discharges the liquid from an outflow port provided to the tank at a constant flow rate. The adjustment tank 12 temporarily internally reserves the liquid that has a non-uniform flow rate and flows from the screen part 11, and discharges the reserved liquid from the outflow port at a constant flow rate.

### (Reaction Tank)

The reaction tank 13 performs, among the predetermined processing performed in the primary processing step, processing that adjusts pH value and the like. Specifically, this reaction tank 13 adjusts the pH value of the liquid fed from the adjustment tank 12. Further, the reaction tank 13 adds a predetermined chemical to the interior thereof, agitates the interior, and flocculates the impurities included in the liquid into a floc state.

### (Aggregate Removal Tank)

The aggregate removal tank 14 performs, among the predetermined processing performed in the primary processing step, processing that removes the aggregates flocculated in the reaction tank 13. This aggregate removal tank 14 settles the aggregates in the lower portion thereof to remove the aggregates from the liquid. Further, the aggregate removal tank 14 removes the aggregates from, the liquid by causing the aggregates to float to the liquid surface using line babbles.

### [Secondary Processing Part]

The secondary processing part 20 biologically processes the liquid after primary processing using microorganisms. The processing step executed by this secondary processing part 20 is the secondary processing step. In the example illustrated in FIG. 2, the secondary processing part 20 comprises the biological treatment tanks 21, 22, 23 including the anaerobic treatment tank 21, the oxygen-free treatment tank 22, and the aerobic treatment tank 23. From upstream, the biological treatment tanks 21, 22, 23 are disposed M the order of the anaerobic treatment tank 21, the oxygen-free treatment tank 22, and the aerobic treatment tank 23.

### (Anaerobic Treatment Tank)

The anaerobic treatment tank 21 performs processing that decomposes soluble organic substances by the microorganisms in the secondary processing step. This anaerobic treatment tank 21 is a treatment tank for decomposing organic matter using a metabolic effect of the microorganisms that requires complete non-existence of oxygen in the liquid after primary processing. Neither dissolved oxygen nor bonded oxygen exists in the interior of the anaerobic treatment tank 21. In this anaerobic treatment tank 21, soluble organic substances are decomposed by the microorganisms, and approximately 10% to approximately 20% of the decomposed organic matter is convened to microorganism bacterial cells, that is, excess sludge.

### (Oxygen-Free Treatment Tank)

The oxygen-free treatment tank 22 performs denitrification processing in an oxygen-free state in the secondary processing step. That is, in the oxygen-free treatment tank 22, denitrifying bacteria, which is one type of facultative anaerobic bacteria, reduces nitrite nitrogen, nitrate nitrogen, and the like to a nitrogen gas and the like using organic matter as the energy source in an oxygen-free state. The oxygen-free treatment tank 22 is a treatment tank in which there is no soluble oxygen, and only the oxygen in nitrous acid or nitric acid exists.

### (Aerobic Treatment Tank)

The aerobic treatment tank 23 performs processing in which the substrate (BOD components in wastewater) and floc configured by a mixed species population of microorganisms are brought into sufficient contact, thereby aerobically oxidizing and decomposing the processing target, with the existence of dissolved oxygen. Aerobic processing can be typically classified into activated sludge processes and biofilm processes. The activated sludge process is a method in which organic substances are oxidized and decomposed with biological floc made to float by aeration. On the other hand, the biofilm process is a method in which biofilm is formed by proliferating microorganisms adhered to carriers, and this biofilm is brought into contact with liquid and oxidized and decomposed. The method implemented by the aerobic treatment tank 23 of the liquid processing device 1 in this embodiment is a type of activated sludge process.

### [Microorganism Optimization Processing Part]

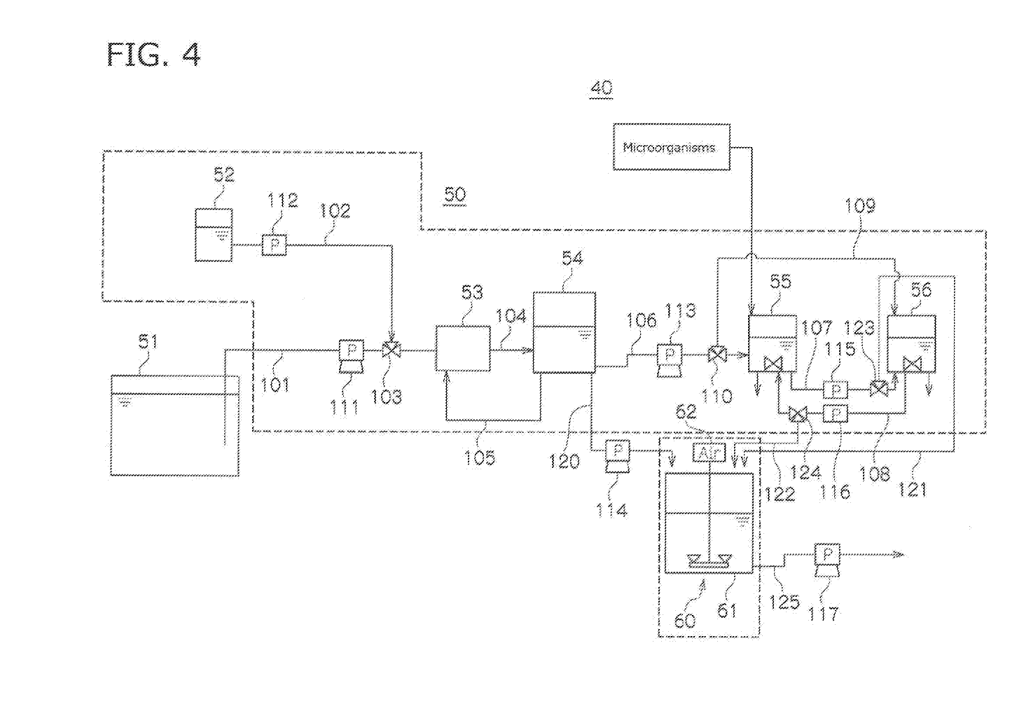

The microorganism optimization processing part 40 executes, a microorganism optimization step that includes a liquid extraction step, a screening step of screening the optimal microorganisms, a culturing step of culturing the optimal microorganisms, and a supply step of supplying the cultured optimal microorganisms into the biological treatment tank, illustrated in FIG. 3. This microorganism optimization processing part 40, as illustrated in FIG. 4, comprises the screening processing part 50 and the culture processing part 60. The screening processing part 50 screens the optimal microorganisms suited for processing the liquid after primary processing. The culture processing part 60 cultures the screened optimal microorganisms.

Note that, as illustrated in FIG. 2, the microorganism optimization processing step performed by the microorganism optimization processing part 40 is individually performed for the microorganisms used in the anaerobic treatment tank 21, the microorganisms used in the oxygen-free treatment tank 22, and the microorganisms used in the aerobic treatment tank 23.

The liquid extraction step (refer to FIG. 3) is a step of directly extracting the stock solution from the liquid handling system or extracting the liquid at an position between the liquid handling system and an inflow port of the biological treatment tank. The screening step is a step of screening the microorganisms suited for secondary processing using the stock solution extracted in the liquid handling system or the liquid extracted at any position between the liquid handling system and the inflow port of the biological treatment tank as the culture substrate. The culturing step is a step of culturing the screened optimal microorganisms.

### <Screening Processing Part>

The screening processing part 50 executes the screening step of screening the optimal microorganisms for the processing of the liquid performed in the secondary processing part 20. The screening processing part 50 uses the stock solution directly extracted from the liquid handling system of a plant or the like that handles the liquid, or the liquid extracted from any position between the liquid handling system and the inflow port of the biological treatment tank as the culture substrate. The details of this screening processing part 50 will now be described with reference to FIG. 4. Note that FIG. 4 illustrates an example of the screening processing part 50. Further, in FIG. 4, the section surrounded by the dashed line is configured as a single unit.

This screening processing part 50 comprises an oxidizing agent reserving tank 52 that reserves oxidizing agent and is for supplying the oxidizing agent to the liquid, a heat sterilization processing pan 53 that sterilizes bacteria included in the liquid by heat, and a sterilized liquid storage tank 54 that reserves the sterilized liquid, and two screening containers 55, 56. The components configured as a single unit for screening are arranged to the order of the heat sterilization processing part 53, the sterilized liquid storage tank 54, and the two screening containers 55, 56, from the upstream side. Note that while the heat sterilization processing part 53 is used as the sterilization processing part in the present embodiment, an ultraviolet sterilization processing part, an ozone sterilization processing part, and other sterilization processing parts may be used in place of the heat sterilization processing part 53.

This screening processing part 50, as illustrated in FIG. 4, is connected to the liquid reserving tank 51, for example, and is configured so that liquid is supplied from this liquid reserving tank 51. This liquid reserving tank 51 is a tank for reserving the liquid and the like discharged from a liquid handling system processing system of a plant or the like that handles the liquid to be processed, for a certain period of time. This liquid reserving tank 51 used is, for example, a tank provided to the liquid handling system of a plant or the like that handles the liquid to be processed. Note that FIG. 4 is a drawing that illustrates one example of supplying the liquid to the screening processing part 50. Extraction of the liquid does not need to be performed by this liquid reserving tank 51. The liquid may be extracted using a pump or the like from a flow path pipe of the liquid handling system processing system, or directly extracted using a pump or the like from the primary processing step. Note that details of extraction of the liquid will be described later.

### (Oxidizing Agent Reserving Tank)

The oxidizing agent reserving tank 52 reserves oxidizing agent for supplying oxidizing agent to the liquid. A pipe 102 is connected to this oxidizing agent reserving tank 52. This pipe 102 is connected to a pipe 101 that connects the liquid reserving tank 51 and the heat sterilization processing part 53 at a position between a pump 111 and the heat sterilization processing part 53. The pipe 102 is connected to the pipe 101 via a switch valve 103. Further, a pump 112 is provided to the pipe 102, and supplies the oxidizing agent of the oxidizing agent reserving tank 52 to the pipe 101. Note that, as the oxidizing agent, for example, a sodium hypochlorite solution, sodium dichloroisocyanurate, chlorine dioxide, hydrogen peroxide, iodine, bromine, or the like is used.

### (Extraction of Liquid)

Here, the position where the liquid used as the culture substrate is extracted will be described.

In this liquid processing device 1, the liquid to be processed is used as the culture substrate of the microorganisms when screening is performed. The liquid used as the culture substrate is (1) the stock solution extracted from the liquid handling system of a plant or the like that handles the liquid to be processed, (2) the liquid undergoing primary processing extracted at any position of the primary processing part 10, or (3) the liquid after primary processing that was subjected to primary processing but not yet incorporated into the secondary processing part 20. Examples of the extraction position when the liquid is extracted at the primary processing part 10 include the adjustment tank 12.

### (Ultraviolet Sterilization Processing Part and Sterilized Liquid Storage Tank)

The heat sterilization processing part 53 is connected to the liquid reserving tank 51 by the pipe 101. This heat sterilization processing part 53 is supplied with the liquid from the liquid reserving tank 51 by the pump 111 provided midway on the pipe 101.

The heat sterilization processing part 53 heats the liquid that serves as the culture substrate, and kills the bacteria. The sterilized liquid storage tank 54 stores the liquid sterilized by the heat sterilization processing part 53. The heat sterilization processing part 53 and the sterilized liquid storage tank 54 are connected by a pipe 104. The liquid is fed from the heat sterilization processing part 53 to the sterilized liquid storage tank 54 through the pipe 104. Further, the heat sterilization processing part 53 and the sterilized liquid storage tank 54 are connected by a pipe 105. The pipe 105 is used as a return line for returning the liquid temporarily fed to the sterilized liquid storage tank 54 to the heat sterilization processing part 53. The liquid is sterilized by being circulated between the heat sterilization processing part 53 and the sterilized liquid storage tank 54. The heat sterilization processing, part 53 sterilizes the liquid, thereby preventing obstruction of microorganism growth when the microorganisms are screened and cultured.

Note that, in the example of the microorganism optimization processing part 40 illustrated in FIG. 4, a sterilization processing step of killing the bacteria included in the liquid serving as the culture substrate is executed using the heat sterilization processing part 53. However, the microorganism optimization processing part 40 may comprise a disinfection device, and thus execute a disinfection processing step. When the disinfection device is included, the microorganism optimization processing part 40 can completely kill or remove the bacteria included in the liquid serving as the culture substrate.

### (Microorganism Screening)

In microorganism screening, the extracted microorganisms are added to the culture substrate and cultured, and the fast-growing microorganisms are selected from among the added microorganisms. Microorganism screening is performed by a plurality of culturing steps in which the culture substrate and the microorganisms are repeatedly housed in the screening containers 55, 56 and the microorganisms are cultured. In the second and subsequent culturing steps of the plurality of culturing steps, the microorganisms and the culture substrate cultured in the previous culturing step are moved to the second screening container 56 that differs from the first screening container 55 used in the previous culturing step, and the microorganisms are cultured. Specifically, using the two screening containers 55, 56 illustrated in FIG. 4, the liquid serving as the culture substrate and the microorganisms are housed in the two screening containers 55, 56, and the microorganisms are cultured for a certain period of time in the interior thereof and screened.

The two screening containers 55, 56 comprise the first screening container 55 disposed on the upstream side, and the second screening container 56 disposed on the downstream side. The first screening container 55 is connected to the sterilized liquid storage tank 54 by a pipe 106. This pipe 106 is provided with a pump 113. In this screening processing part 50, the pump 113 is activated when necessary, and the sterilized liquid stored in the sterilized liquid storage tank 54 is supplied to the first screening container 55.

The first screening container 55 and the second screening container 56 are connected by two pipes 107, 108. Of the two pipes 107, 108, the one pipe 107 is used when the liquid serving as the culture substrate and the microorganisms housed in the first screening container 55 are supplied from the first screening container 55 to the second screening container 56. The other pipe 108 is used when the liquid serving as the culture substrate and the microorganisms housed in the second screening container 56 are returned from the second screening container 56 to the first screening container 55.

Pumps 115, 116 are provided to the pipes 107, 108, respectively. The pump 115 provided to the pipe 107 is activated when necessary, thereby supplying the liquid serving as the culture substrate and the microorganisms housed in the first screening container 55 from the first screening container 55 to the second screening container 56. In contrast, the pump 116 provided to the pipe 108 is activated when necessary, thereby returning the liquid serving as the culture substrate and the microorganisms housed in the second screening container 56 from the second screening container 56 to the first screening container 55. Further, pipes 121, 122 that connect the pipes 107, 108 and the culture processing part 60 described later are respectively connected to the pipes 107, 108 via switch valves 123, 124.

The second screening container 56 is further connected to the pipe 106 on the downstream side of the pump 113 by a pipe 109. The pipe 109 is connected to the pipe 106 via a valve 110. This pipe 109 is used when the liquid stored in the sterilized liquid storage tank 54 is supplied directly to the second screening container 56 without passing through the first screening container 55.

The screening step executed, by the screening processing part 50 comprises a plurality of culturing steps of repeatedly housing the culture substrate and the microorganisms in the screening containers 55, 56 and culturing the, microorganisms, as previously described, in the third and subsequent culturing steps, the microorganisms and the culture substrate cultured in the previous culturing step are moved to the second screening container 56 that differs from the first screening container 55 used in the previous culturing step, and the microorganisms are cultured. In the example illustrated in FIG. 4, each of the culturing steps is performed as follows using the first screening container 55 and the second screening container 56.

First, the sterilized liquid is fed from the sterilized liquid storage tank 54 to the first screening container 55. This sterilized liquid is used as the culture substrate. Further, the microorganisms are added to the first screening container 55. In the screening step performed using this screening processing part 50, upper-level fast-growing microorganisms are screened from among the added microorganisms. The added microorganisms are extracted from the biological treatment tanks 21 22, 23. However, the added microorganisms are not limited to those extracted from the biological treatment tanks 21, 22, 23, and may be extracted from an external environment other than the biological treatment tanks 21, 22, 23.

Note that “an external environment other than the biological treatment tanks 21, 22, 23” refers to locations where steps excluding the biological processing steps performed by the liquid processing device 1 are executed, and locations other than the liquid processing device 1. For example, microorganisms extracted from the first processing part that executes steps other than the biological processing steps or the like, microorganisms extracted in locations completely different from the liquid processing device 1, or commercialized microorganisms may be used.

In the first culturing step of the screening step, the microorganisms and the sterilized liquid are housed in the first screening container 55, and the microorganisms are allowed to grow for a certain period of time. After this first culturing step is completed, the grown microorganisms are moved to the second screening container 56, and the next second culturing step is executed. In the second culturing step, the sterilized liquid fed from the sterilized liquid storage tank 54 via the pipe 109 and the grown microorganisms are housed in the second screening container 56, and the microorganisms grow for a certain period of time in the second screening container 56. Next, the third culturing step is executed. In the third culturing step, the microorganisms grown in the second culturing step are returned once again to the first screening container 55, the sterilized liquid is fed from the sterilized liquid storage tank 54 to the first screening container 55, and the microorganisms grow in the first screening container 55.

In this screening step, the sterilized liquid serving as the culture substrate and the microorganisms are mutually housed between the first screening container 55 and the second screening container 56. The microorganisms grow in the screening containers 55, 56, thereby screening the upper-level fast-growing microorganism from among the microorganisms initially added. Note that each of the culturing steps is executed from several hours to about 24 hours.

### <Culture Processing Part>

The culture processing part 60 is a processing part for culturing the microorganisms screened by the screening processing part 50 in large quantities. The culture processing part 60 comprises a culture tank 61 having a capacity corresponding to the situation, and an air pump 62. The air pump 62 feeds air to the culture tank 61 when the microorganisms used in the aerobic treatment tank 23 are cultured.

The culture processing part 60 is connected to the sterilized liquid storage tank 54 by a pipe 120. The pipe 120 is provided with a pump 114. Further, the pipes 121, 122 are connected to the culture processing part 60. The pipes 121, 122 are connected to the pipes 107, 108 via the switch valves 123, 124, respectively. Moreover, the culture processing part 60 comprises a pipe 125. The pipe 125 connects the culture tank 61 and the biological treatment tanks 21, 22, 23. This pipe 125 is provided with a pump 117. The cultured microorganisms are supplied to any one of the anaerobic treatment tank 21, oxygen-free treatment tank 22, or the aerobic treatment tank 22 constituting the biological treatment tanks 21, 22, 23 via the pipe 125 by the pump 117.

The sterilized liquid is led from the sterilized liquid storage tank 54 to this culture tank 61 of the culture processing part 60. Supply of the sterilized liquid from the sterilized liquid storage tank 54 to the culture tank 61 is performed upon activation of the pump 114 provided to the pipe 120. This sterilized liquid is used as the culture substrate. Further, microorganisms screened by the screening processing part 50 are added to the culture tank 61. The microorganisms are added by being supplied from the first screening container 55 or the second screening container 56 to the culture tank 61. When the microorganisms are added, the switch valve 123 or the switch valve 124 is opened and the pump 115 or the pump 116 is activated. In this culture processing part 60, the sterilized liquid and the screened microorganisms are housed in the culture tank 61, and the optimal microorganisms are cultured in large quantities over a period of from several hours to about 24 hours. Further, the microorganisms may be cultured continually. Continuous culturing of the microorganisms is performed by continuously supplying a certain amount of culture substrate from the sterilized liquid storage tank, and continuously supplying the preparation to the biological treatment tank.

The liquid processing device 1 executes a microorganism supply step of supplying microorganisms cultured by the culture processing part 60 to the biological treatment tanks 21, 22, 23. This microorganism supply step is performed by supplying the microorganisms housed in the culture tank 61 to the biological treatment tanks 21, 22, 23. When the microorganisms are supplied, the pump 117 provided to the pipe 125 is activated.

### (Timing Determination Means and Timing Determination Step)

In the above, the basic configuration of the microorganism optimization processing part 40 and the basic step performed by the microorganism optimization processing part 40 were described. This liquid processing device comprises timing determination means for executing a timing determination step of determining the timing at which the aforementioned screening step is executed. Specifically, a controller 80 illustrated in FIG. 2 determines the timing.

The timing determination means promotes execution of the screening step. The timing at which execution is promoted is periodic or when a given physical property determined in advance in the stock solution or the liquid extracted in the liquid extraction step reaches a control level set in advance. In this liquid processing device 1, the timing determination means determines the timing at which the screening step is to be executed and the screening step is executed in an appropriate time period, thereby avoiding various risks.

That is, a daily load fluctuation of the stock solution of the wastewater discharged from a plant or the like is severe. Techniques for biologically processing such wastewater and the like are always plagued with risks of a decrease in processing capacity and incomplete processing such as described below, regardless of industry and region.

### (1) Risk of Environmental Factor Fluctuation

Environmental factors fluctuate along with changes in season, sudden temperature changes, and the like. Such risk can be avoided as follows. That is, microorganisms compatible with the drainage environment and temperature changes are obtained by selecting the optimal microorganisms at the time while matching the culture temperature with the water temperature.

(2) Risk of Fluctuation in Processing Conditions Associated with Changes or Modifications in Liquid Processing Facility Conditions

The preprocessing conditions of processing that uses microorganisms may change. Such changes include, for example, defects or deterioration that occur in equipment for implementing preprocessing steps. Such risk may be resolved or reduced by periodically surveying the conditions of the liquid to be processed and selecting microorganisms applicable to the conditions when a change in water quality is detected.

### (3) Risk of Collapse of Balance in Microorganism Tank

During the period of long-term shutdown of to plant line, new liquid to be processed may no longer exist in the microorganism tank. In such a case, the balance in the microorganism tank collapses. Such risk can be avoided as follows. That is, the technique for avoiding risk is screening, culturing, and adding microorganisms adapted to the liquid to be processed when the liquid processing device is started. This technique for avoiding risk makes it possible to start the processing device in a short period of time.

(4) Risk Resulting from Plant Operation Factors

In the plant serving as the liquid handling system, the liquid to be processed may change in quality due to periodic cleaning, increases and decreases in production, and the like. Changes in the quality of the liquid overload the microorganisms or make the microorganisms enter an oligotrophic state. Thus, even if liquid having properties different than usual is discharged, the risk of abnormalities in the biological treatment tank is resolved or reduced by surveying, the water quality and, when the water quality changes, selecting microorganisms that match the new conditions.

### (5) Unanticipated Risk

Unanticipated recalcitrant substances and substances having biological growth inhibitive capability (antibiotics and the like) may flow into the liquid to be processed. When recalcitrant substances and substances having biological growth inhibitive capability flow into the liquid to be processed either temporarily or for the medium or long term, the processing capacity of the microorganism tank is maintained by periodically selecting the optimal microorganisms.

In a case where the wastewater of a liquid handling system having a constantly changing environment is processed by promoting the periodic execution of screening by the timing processing determination means, this liquid processing device 1 can process the wastewater using microorganisms applicable to such changes. On the other hand, in a case where execution of the screening step is promoted when a given physical property determined in advance in the stock solution or liquid extracted in the liquid extraction step reaches a control level set in advance, this liquid processing device 1 can appropriately respond to sudden changes as well.

When screening is performed periodically, the screening is preferably performed every one to two weeks.

Note that examples of means for detecting the water quality include sensors for detecting the BOD, COD, total organic carbon (TOC), turbidity, and the like.

Further, the liquid processing device 1 comprises the timing determination means described above, thereby allowing the liquid processing device 1 to select either microorganisms optimized in a screening step performed in the past or microorganisms optimized in the screening step currently performed, whichever is suited for processing the current liquid subjected to primary processing.

That is, the liquid processing device 1 can determine the timing at which the screening step is performed by the timing determination means a plurality of times. The screening step is executed a plurality of times in accordance with the number of times the timing was determined.

The screening processing part 50 compares the following microorganisms. One is the microorganisms screened in a most recent screening step executed at a most recent timing determined by the timing determination means. Another is the microorganisms screened in the screening step executed earlier than the most recent timing. Then, the screening processing part 50 compares the two and selects which of these microorganisms is appropriate for processing the current liquid subjected to primary processing.

Note that the microorganisms screened M the earlier screening step are stored by a method suited for the microorganisms. When the microorganisms screened in the new screening step and the microorganisms screened in the earlier screening step are compared, the microorganisms screened in the earlier screening step are used as the stored microorganisms.

With the liquid processing device 1 executing the above steps, the liquid processing device 1 can select the most appropriate microorganisms and process the liquid after primary processing.

### [Tertiary Processing Part]

The tertiary processing part 30 executes a tertiary processing step of processing content and the like not processed by the primary processing part 10 or the secondary processing, part 20. This tertiary processing part 30 comprises, for example, a flocculent settling tank 31 and a sand filter 32. Note that the tertiary processing part illustrated in FIG. 2 is an example. The tertiary processing part 30 can also be provided with processing means other than the flocculent settling tank 31 and the sand filter 32. For example, the tertiary processing part 30 may be provided with processing means for oxidizing liquid.

The tertiary processing part 30 feeds the liquid processed by'the secondary processing part 20 into the flocculent settling tank 31, for example, and flocculates and settles suspended matter. Subsequently, the tertiary processing part 30 removes impurities by means such as flocculating, settling, floating, filtering, adsorbing, and oxidizing, in accordance with the processing intent. Thus, the tertiary processing part 30 settles the floc (clumps of microorganisms) included in the processed water after biological processing in the flocculent settling tank 31, and returns a portion thereof to the anaerobic treatment tank 21 as returned sludge. The remaining sludge settled in the flocculent settling tank is fed to the sludge discharging part 70 described later.

The sand filter 32 is processing means for removing impurities and the like included in the liquid processed in the flocculent settling tank 31. This sand filter 32 filters the impurities and the like included in the liquid by passing the fed liquid through sand filled in the interior thereof. The liquid processed by this sand filter 32 is released as illustrated in FIG. 2. Note that the liquid processed by the tertiary processing part 30 is reused as necessary.

The sludge produced by the primary processing part 10, the secondary processing part 20, and the tertiary processing part 30 is fed to the sludge discharging part 70. The sludge discharging part 70 comprises a sludge tank 71 where the sludge is collected, and a dehydrator 72. The sludge is temporarily collected in the sludge tank 71. The sludge stored in the sludge tank 71 contains moisture. The sludge that contains moisture is fed from the sludge tank to the dehydrator 72, and is dehydrated by the dehydrator 72. The dehydrated sludge is appropriately processed as industrial waste by being discarded or incinerated.

### EXAMPLES

In the following, the present invention is described in further detail using examples and comparative examples.

### [Test 1]

In test 1, microorganisms having a high COD removal rate were optimized using the liquid processing device 1 illustrated in FIG. 2. The liquid handling system was a soap plant. The culture substrate used for screening and culturing the optimal microorganisms was obtained by heating the wastewater of the soap plant prior to activated sludge processing to 121° C., and disinfecting the wastewater for 15 minutes. Further, the microorganisms used for screening were extracted from the soap plant. Specifically, a carrier in the activated sludge extracted from the soap plant was used. In test 1, the COD removal capability was examined using the microorganisms cultured based on the conditions described above.

### Example 1

In example 1, microorganisms were screened by the following steps.

First, one activated sludge carrier into which the microorganisms extracted in the soap plant were added was housed in the screening container 55 along with 50 mL of the culture substrate. The temperature in the interior of the screening container 55 was 23° C. The activated sludge carrier into which the microorganisms were added was a cube having 1-cm sides.

Next, with the screening container 55 shaken 70 times per minute, shake culturing was performed repeatedly for 24 hours. Note that, after completion of one shake culturing, culturing was similarly performed by subculturing 0.5 mL of the cultured liquid in 50 mL of a new culture substrate, and this series of tasks was continued for nine days.

Subsequently, the type of bacteria grown on a plate medium was observed with the naked eye. Further, a simplified property inspection was also implemented using an identification kit.

The microorganisms obtained by this screening were shewanella putrefaciens and agrobacterium tumefaciens.

Then, among the obtained microorganisms, culturing was performed using the shewanella putrefaciens, which had a large number of bacteria, as the optimal microorganisms. The optimal microorganisms and activated sludge were added to 1 L of culture substrate so that the number of added bacterial cells was approximately 1×10⁵ CFU/mL and, with the mixture shaken 70 times per minute, shake culturing was performed for four hours at a room temperature of 23° C.

Note that “CFU/mL” is a colony-forming unit and, in microbiology, expresses the number of colonies formed when a certain amount of microorganisms (bacteria and the like) are spread across a solid medium in which the microorganisms grow.

### Example 2

The microorganism in example 2 were also microorganisms having a high COD removal rate. In example 2, a soap plant was selected as the liquid handling system, similar to example 1.

In the step of screening and culturing microorganisms in example 2, three activated sludge carriers, into which microorganisms extracted in the soap plant were added, were added to 50 mL of a culture substrate. Note that the used culture substrate and the steps other than microorganism screening and culturing were the same as those in example 1. The microorganisms obtained by this screening were the same as those in example 1.

Then, culturing was performed using shewanella putrefaciens as the optimal microorganisms, similar to example 1.

### Example 3

The microorganisms in example 3 were also microorganisms having a high COD removal rate. In example 3, a soap plant was selected as the liquid bundling, system, similar to example 1. Then, the microorganisms having a high COD removal rate were optimized using scum extracted from the soap plant.

In the step of screening and culturing microorganisms in example 3, 0.15 grams of scum, into which microorganisms extracted in the soap plant were added, were added to 50 mL of a culture substrate. Note that the used culture substrate and the steps other than microorganism screening and culturing were the same as those in example 1. The microorganisms obtained by this screening were the same as those in example 1.

Then, culturing was performed using shewanella putrefaciens as the optimal microorganisms, similar to example 1.

### Example 4

In example 4, with the assumption of use of optimal microorganisms mixed together with the activated sludge, a mixed sample obtained by adding the activated sludge and the optimal microorganisms obtained through the same steps as in example 1 in an amount of approximately 1×10⁵ CFU/mL each was cultured for four hours using the same method as in example 1.

### Comparative Example 1

In the comparative example, a general activated sludge was used.

### <COD Removal Capacity Test>

A COD removal capacity test was conducted using the optimal microorganisms obtained in examples 1 to 3, and the microorganisms in example 4. The test was performed by measuring the change in COD concentration, and calculating the COD removal rate. Note that the concentration of added bacterial cells needed to be made to the same degree in order to compare the optimal microorganisms and general activated sludge. Thus, the number of bacteria in the optimal microorganisms and the activated sludge solution was measured, and the concentrations of the bacterial cells in both were made the same in advance.

The COD concentration measurement was, performed on the stock solution discharged from the soap plant. The COD concentration of the stock solution to be measured was initially 140 mg/L.

TABLE 1

| | COD (mg/L) | COD Removal Rates (%) |
|---|---|---|
| Example 1 | 91 | 35 |
| Example 2 | 91 | 35 |
| Example 3 | 91 | 35 |
| Example 4 | 87 | 38 |
| Comparative | 135 | 3.6 |
| Example | | |

Table 1 summarizes the COD measurement results. As shown in Table 1, the COD concentration only decreased to 135 mg/L and the COD removal rate was merely 3.6% with the activated sludge based processing. In contrast, the COD concentration decreased to 91 mg/L and the COD removal rate was 35% in examples 1 to 3 that used the optimal microorganisms. Further, the COD concentration decreased to 87 mg/L and the COD removal rate was 38% even in example 4 in which culturing was performed using the mixed sample of optimal microorganisms and activated sludge. In contrast, the COD removal rate was 38% with the activated sludge based processing.

From the results of this test, it was clearly understood that, according to examples 1 to 3, it is possible to reduce COD in a short period of time. Further, it was clearly understood that, even when existing activated sludge was added as in example 4, the COD removal rate did not decrease, and thus existing activated sludge was found not to compete with the optimal microorganisms or obstruct processing.

### [Test 2]

In, test 2, the screening of microorganisms was performed using a screening tester manufactured by the inventors of the present invention, and the degree to which the screened microorganisms could remove total organic carbons (hereinafter referred to as “TOC”) was measured. Specifically, in test 2, the TOC removal rate was found. Note that “TOC” expresses in carbon the total amount of organic matter that can be oxidized in water.

The basic structure of the screening tester 40 used in test 2 was the same as that of the device illustrated in FIG. 4. Thus, the reference number of the screening tester used is 40. In test 2, two screening containers including the first screening container 55 and the second screening container 56 were used to culture the microorganisms. Specifically, the microorganisms were mutually transferred between the first screening container 55 and the second screening container 56, and screening was executed by culturing the optimal microorganisms so as to gradually increase the number of microorganisms in each of the screening containers 55, 56.

### Example 5

In example 5, an activated sludge extracted from the soap plant was used. Further, plant wastewater prior to being processed by the activated sludge was heated and sterilized for 10 minutes at 95° C. and used as the culture substrate. Then, the activated sludge described above was screened, thereby obtaining the microorganisms in example 5, which were microorganisms having a high TOC removal rate.

A specific description of the screening will now be given. In this test 2, the activated sludge extracted from the soap plant was screened using the screening tester 40, and a test for obtaining microorganisms having a high TOC removal rate was performed. The microorganisms were cultured for 24 hours upon injecting 1 L of the culture substrate into the first screening container 55 and adding 1 mL of the activated sludge. The conditions when the microorganisms were cultured included an ambient temperature of 25° C., an aeration amount of 1 L/minute, and an agitation of 200 revolutions per minute (200 rpm) of the interior of the first screening container 55.

After the microorganisms were cultured using the first screening container 55, the cultured liquid in Which the microorganisms were cultured was transferred to the second screening container 56, and culturing was continued. Am this time, 1 L of the culture substrate was injected and 1 mL of the cultured liquid was transferred from the first screening container 55 to the second screening container 56 and subculturing was performed. After subculturing, culturing was performed under the same conditions as the conditions described above in the second screening container 56. Such a series of operations in which the cultured liquid is transferred between the first screening container 55 and the second screening container 56 was repeated four times. Example 5 was obtained by executing the above operations. Then, the TOCs of the culture substrate before the fourth culturing started and after the fourth culturing ended were measured and the removal rate was found. Further, the number of general bacteria upon culturing completion was measured.

### Comparative Example 2

In comparative example 2, a generally commercialized microbial preparation was used. In comparative example 2, the microbial preparation was added to 1 L of the culture substrate, and the number of added bacterial cells was set to approximately 1×10⁵ CFU/mL. The microorganisms were cultured for 24 hours upon injecting 1 L of the culture substrate into the first screening container 55 and adding 1 mL of the activated sludge.

### Comparative Example 3

In comparative example 3, the microorganisms were cultured by performing the same operations with the activated sludge as those in comparative example 2.

In this comparative example 2, the TOCs of the culture substrate before culturing started and after culturing ended were measured and the removal rate found. Further, the number of general bacteria alter culturing completion was measured.

### [Test Results]

To verify that the culture substrate used for screening was sterilized, the culture substrate was sterilized for 10 minutes at 95° C. and the number of general bacteria was measured three times. As a result, growth of bacteria was not found on the plate medium in any of the measurements. Based on this result, obstruction of the screening step by contamination is considered prevented.

Table 2 shows the measurement results of the TOC value. The values shown in Table 2 are the TOC values before culturing started and alter culturing ended in example 5, comparative example 2 (microbial preparation), and comparative example 3 (activated sludge).

TABLE 2

| | TOC (mg/L) | | |
|---|---|---|---|
| | 0 hours | 24 hours | Removal Rates (%) |
| Example 5 | 644 | 508 | 21 |
| Comparative Example 2 | 648 | 557 | 14 |
| Comparative Example 3 | 627 | 555 | 11 |

As shown in Table 2. before the microorganisms were cultured, that is, at a culture time of 0 hours, the TOC values of example 5, comparative example 2, and comparative example 3 were 644 mg/L, 648 mg/L, and 627 mg/L, respectively. Then, the TOC values of example 5, comparative example 2, and comparative example 3 after the microorganisms were cultured and 24 hours passed were 508 mg/L, 557 mg/L and 555 mg/L respectively. The TOC removal rates of example 5, comparative example 2, and comparative example 3 were 21%, 14%, and 11%, respectively. That is, the TOC removal rate of example 5, which were the optimal nncroorganisms, was the highest at 21%, the TOC removal rate of the microbial preparation was the next highest at 14%, and the TOC removal rate of the activated sludge was the lowest at 11%. As understood from the results shown in this Table 2, it was found that when the screening method according to the present invention was performed, the microorganisms that effectively remove TOC could be cultured compared to when the microbial preparation was used and when the activated sludge was used. Note that examples of future problems to be addressed include the identification of the specific microorganisms cultured.

Table 3 summarizes the number of general bacteria after the microorganisms were cultured for 24 hours.

TABLE 3

| | Numbers of General Bacteria (CUF/mL) |
|---|---|
| Example 5 | 3.6 × 10⁹ |
| Comparative Example 2 | 4.0 × 10³ |
| Comparative Example 3 | 1.6 × 10⁷ |

As shown in Table 3. the number of general bacteria after the microorganisms were cultured for 24 hours was 3.6×10⁹ for example 5, 4.0×10³ for comparative example 2, and 1.6×10⁷ for comparative example 3, and the number of general microorganisms inside the container in which, the optimal microorganisms serving as example 5 were cultured was found to be the largest.

That is, as shown in Table 3, it was found based on the results of this test 2 that, when screening was implemented by the method adapted to obtain the microorganisms in this example 5, optimal microorganisms having a high processing capacity with respect to the culture substrate were selected over the microbial preparation and the activated sludge used in comparative example 2.

As also understood from the above test results, when the activated sludge was extracted from a facility or the like to be processed and the microorganisms included in the extracted activated sludge were screened, thereby culturing the optimal microorganisms, COD processing and TOC processing were found to have considerably high capacities compared to when the general activated sludge was used and when the microbial preparation was added to L of the culture substrate and the microorganisms were cultured.

### DESCRIPTIONS OF REFERENCE NUMERALS

1 Liquid processing device
10 Primary processing part
11 Screen part
12 Adjustment tank
13 Reaction tank
14 Aggregate removal tank
20 Secondary processing part
21 Anaerobic treatment tank (biological treatment tank)
22 Oxygen-free treatment tank (biological treatment tank)
23 Aerobic treatment tank (biological treatment tank)
30 Tertiary processing part
31 Flocculent settling tank
32 Sand filter
40 Microorganism optimization processing part
50 Screening processing part
51 Liquid reserving tank
52 Oxidizing agent reselling tank
53 Heat sterilization processing part
54 Sterilized liquid storage tank
55 First screening container
56 Second screening container
60 Culture processing part
61 Culture tank
62 Air pump
70 Sludge discharging part
71 Sludge tank
72 Dehydrator
80 Controller
101, 102, 104, 105, 106, 107, 108 Pipe
103 Switch valve
111, 112, 113, 114, 115, 116, 117 Pump
120, 121, 122, 125 Pipe
110, 123, 124 Switch valve

1. A liquid processing method, comprising:
processing a liquid in a biological treatment tank that houses microorganisms, and
processing a liquid discharged from a liquid handling system that handles the liquid, the liquid processing method further comprising:
optimizing microorganisms in the biological treatment tank to obtain optimal microorganisms;
the microorganism optimization comprising:
performing a liquid extraction by extracting a liquid comprising a stock solution discharged from the liquid handling system;
screening the optimal microorganisms suited for processing performed in the biological treatment tank with the liquid comprising the stock solution as a culture substrate; and
culturing the screened optimal microorganisms.

2. The liquid processing method according to claim 1, further comprising:
supplying the cultured optimal microorganisms into the biological treatment tank.

3. The liquid processing method according to claim 1, further comprising:
a primary processing of incorporating a stock solution discharged from the liquid handling system and performing predetermined processing; and
a secondary processing of processing a liquid after primary processing that was subjected to the predetermined processing in the biological treatment tank;
the liquid extraction directly extracting the stock solution from the liquid handling system or extracting the liquid at any position between the liquid handling system and an inflow port of the biological treatment tank.

4. The liquid processing method according to claim 1, wherein:
the microorganism optimization comprises a microorganism extraction of extracting microorganisms from, among the biological treatment tank and an external environment other than the biological treatment tank, at least the biological treatment tank; and
the screening adds microorganisms extracted in the microorganism extraction to the culture substrate.

5. The liquid processing method according to claim 4, wherein
the screening adds microorganisms extracted in the microorganism extraction to the culture substrate, cultures the microorganisms, and selects the fast-growing microorganisms from among the added microorganisms.

6. The liquid processing method according to claim 1, wherein
the microorganism optimization further comprises a timing determination of determining the timing at which the screening step is executed.

7. The liquid processing method according to claim 6, wherein
the timing determination promotes execution of the screening periodically or when a given physical property determined in advance in the stock solution or the liquid extracted in the liquid extraction reaches a control level set in advance.

8. The liquid processing according to claim 6, wherein:
the microorganism optimization can be executed a plurality of times by the timing determined in the timing determination; and
the screening compares microorganisms screened in a most recent screening executed at a most recent timing determined in the timing determination, and microorganisms cultured in the microorganism optimization executed earlier than the most recent timing, and comprises a microorganism selecting of selecting microorganisms suited for the secondary processing at the most recent timing.

9. The liquid processing method according to claim 1, wherein
the screening comprises a plurality of culturing operations of repeatedly housing the culture substrate and the microorganisms in a screening container and culturing the microorganisms.

10. The liquid processing method according to claim 1, wherein
the microorganism optimization further comprises a disinfection processing or a sterilization processing that disinfects or sterilizes the stock solution or the liquid extracted in the liquid extraction.

11. A liquid processing device that comprises a biological treatment tank that houses microorganisms and is for processing a liquid, and processes a liquid discharged from a liquid handling system that handles the liquid, the liquid processing device further comprising:
a microorganism optimization processing part for optimizing microorganisms used in the biological treatment tank to obtain optimal microorganisms;
the microorganism optimization processing part comprising:
a liquid extracting part that extracts a liquid that includes a stock solution discharged from the liquid handling system;
a screening processing part that screens the optimal microorganisms suited for processing performed in the biological treatment tank using the liquid that includes the stock solution as a culture substrate; and
a culture processing part that cultures the screened optimal microorganisms.

12. The liquid processing device according to claim 11, further comprising
a supply part that supplies the cultured optimal microorganisms into the biological treatment tank.

13. The liquid processing device according to claim 11, further comprising:
a primary processing part that incorporates a stock solution discharged from the liquid handling system, performs predetermined processing, and supplies the liquid after the predetermined processing to the biological treatment tank;
the liquid extracting part directly extracting the stock solution from the liquid handling system or extracting the liquid at any position between the liquid handling system and an inflow port of the biological treatment tank.

14. The liquid processing device according to claim 11, wherein
the screening processing part comprises a disinfection processing part or a sterilization processing part that sterilizes or disinfects the stock solution or the liquid.

15. The liquid processing method according to claim 7, wherein:
the microorganism optimization can be executed a plurality of times by the timing determined in the timing determination step; and
the screening compares microorganisms screened in a most recent screening step executed at a most recent timing determined in the timing determination, and microorganisms cultured to the microorganism optimization executed earlier than the most recent timing, and comprises a microorganism selecting of selecting microorganisms suited for the secondary processing step at the most recent timing.
